(19) United States
(12) Patent Application Publication
(10) Pub. No.: US 2017/0265828 A1
(43) Pub. Date: Sep. 21, 2017

(54) RADIATION IMAGING APPARATUS, AND INSERTION STATE DETERMINATION METHOD

(71) Applicant: CANON KABUSHIKI KAISHA, Tokyo (JP)

(72) Inventor: Osamu Tsujii, Kawasaki-shi (JP)

(21) Appl. No.: 15/433,546

(22) Filed: Feb. 15, 2017

(30) Foreign Application Priority Data
Mar. 16, 2016 (JP) 2016-052833

(51) Int. Cl. A61B 6/00; A61B 6/04; A61B 5/00; A61B 6/03

(52) U.S. Cl. CPC A61B 6/502; A61B 6/032; A61B 6/0414; A61B 6/4085; A61B 6/0492; A61B 5/0091; A61B 6/544; A61B 2562/0247

(57) ABSTRACT

Provided is a medical image imaging apparatus that images a medical image of a breast, including a determination unit configured to determine an insertion state of the breast based on a form of the breast inserted into an imaging area for the medical image from an insertion portion.

### BACKGROUND OF THE INVENTION

Field of the Invention

The present invention relates to a radiation imaging apparatus for imaging a radiological image of a breast, an insertion state determination method.

Description of the Related Art

A mammogram apparatus is normally used as an X-ray inspection apparatus for breast cancer. However, it is known that in the case of taking a mammogram of a dense breast (a breast with many mammary glands), a lesion portion and a mammary gland structure overlap each other, which leads to deterioration in the sensitivity and specificity of lesion detection. Tomosynthesis and CBCT apparatus exclusively for breast imaging have received attention as techniques for compensating for the shortcomings of the mammogram. These apparatuses have a feature that a 3D image of a breast is provided so that the lesion portion and the mammary gland structure can be observed separately.

In the case of imaging a breast by the CBCT apparatus exclusively for breast imaging, it is necessary to manage a blind area (imaging inability area). In the case of CC imaging and MLO imaging of a mammogram and “Breast CT” imaging of a prone position type as disclosed in Japanese Patent Application Laid-Open No. 2010-68929, an imaging area (or a blind area) is managed by an engineer.

Not only in breast X-ray imaging apparatuses, but also in many of the general X-ray imaging apparatuses, an X-ray irradiation field is projected using a projection optical system. Similarly, in X-ray CT apparatuses, an imaging position (slice position) is illuminated with visible light.

There is disclosed a technique relating to an X-ray CT apparatus including: an X-ray generation unit that generates a cone beam; a detection unit having a plurality of rows of detection elements that detect an X-ray; and a light projecting unit that projects light indicating a boundary between detected ranges of the detection unit in the axis direction of a subject onto the subject (see Japanese Patent Application Laid-Open No. 2015-139534).

Further, there is disclosed an X-ray imaging apparatus having a structure in which in a breast imaging CBCT, an X-ray generation apparatus and an X-ray detector are vertical to a top board, are opposed to each other with a rotation axis A passing through an opening from which a breast is set, and rotate about the rotation axis (see Japanese Patent Application Laid-Open No. 2010-68929). This apparatus generates an X-ray tomographic image using an X-ray transparent image, which is obtained by irradiating an X-ray cone beam for X-ray tomographic image, while the apparatus-ray generation apparatus and the X-ray detector rotate. In the apparatus, an X-ray imaging apparatus is disposed at an arrangement position determined based on the X-ray tomographic image, and provides an X-ray transparent planar image of a breast that is obtained by the X-ray cone beam for planar image.

In the mammogram or CBCT exclusively for breast imaging, it is well known that a blind area, in which a radiological image is not imaged, is generated. In the mammogram, two imaging views of a CC view and an MLO view are used to complement the blind area. In practice, blind areas are managed through manipulation by an engineer.

In a prone position type breast CBCT as disclosed in Japanese Patent Application Laid-Open No. 2010-68929, the blind areas are more likely to be generated than in the mammogram. Particularly, it is difficult to insert a breast into an axilla-side area or an axilla area of the breast, so that the blind areas are likely to be generated in these areas. The prone position type breast CBCT is designed to be able to image an area including a breast bone by causing the body of a subject to be tilted by the weight of the subject so that the blind areas can be reduced. However, tilting the body is a physical burden for the subject, and imaging of the area including the breast bone is not preferable in terms of imaging exposure.

On the other hand, in order to improve a throughput and reduce a physical burden of a subject during imaging, an upright breast CT (upright breast CBCT) has been devised. In the upright breast CBCT, the body of the subject cannot be tilted using the weight of the subject to insert the breast into an insertion portion, unlike in the prone position type, so that the breast cannot be sufficiently inserted (i.e., the insertion state is unsuitable) in some cases and the blind area varies depending on the insertion state. The variation in the blind area causes a problem that it is difficult to image an intended area. Another problem is that the variation in the blind area in the upright breast CBCT is larger than the variation of the blind area in the mammogram that is taken by fixing the breast.

### SUMMARY OF THE INVENTION

The present invention determines an insertion state (an insertion position or an insertion direction) of a breast to manage an imaging area or a blind area during imaging of a radiological image of the breast.

A medical image imaging apparatus according to the present invention is a medical image imaging apparatus that images a medical image of a breast and includes a determination unit configured to determine an insertion state of the breast based on a form of the breast inserted into an imaging area for the medical image from an insertion portion.

Further features of the present invention will become apparent from the following description of exemplary embodiments with reference to the attached drawings.

### BRIEF DESCRIPTION OF THE DRAWINGS

FIG. 1 is a view illustrating an example of a radiation imaging system according to a first embodiment.

FIG. 2 is a view illustrating the radiation imaging system according to the first embodiment as viewed from a mammogram imaging side.

FIG. 3 is a view illustrating a position of a patient during CBCT imaging in the radiation imaging system according to the first embodiment.

FIG. 4 is a diagram illustrating an example of the configuration of the radiation imaging system according to the first embodiment.

FIG. 5A is a diagram illustrating an example of divided areas of a breast.

FIG. 5B is a diagram of the breast when the breast is observed from the leg portion side of the subject.

FIGS. 5C and 5D are diagrams each illustrating an example of the insertion state of the breast.

FIGS. 6A, 6B and 6C are diagrams each illustrating the generation of a blind area according to an insertion state of the breast.

FIG. 7Aa is a diagram illustrating an example of the insertion state of the breast.

FIG. 7Ab is a diagram illustrating an example of a projection optical system.

FIGS. 7Ba and 7Bb are diagrams each illustrating an example in which an imaging area is displayed on the perimeter of an inserted breast.

FIGS. 7Ca and 7Cb are diagrams each illustrating a case where inserted breasts are a right breast and a left breast.

FIG. 7D is a diagram illustrating an example in which the imaging area is not displayed in a lower area of the inserted breast.

FIGS. 7Ea and 7Eb are diagrams each illustrating a modified example of the radiation imaging system according to the first embodiment.

FIG. 8 is a diagram illustrating an example of a radiation imaging system according to a second embodiment.

FIG. 9A is a diagram illustrating a modified example of the radiation imaging system according to the second embodiment using a proximity sensor.

FIG. 9B is a diagram illustrating a modified example of the radiation imaging system according to the second embodiment using a contact sensor (pressure sensor).

FIGS. 10A and 10B are diagrams each illustrating an example in which the insertion state of the breast is determined based on the volume of an inserted breast or a non-inserted breast.

### DESCRIPTION OF THE EMBODIMENTS

Preferred embodiments of the present invention will now be described in detail in accordance with the accompanying drawings.

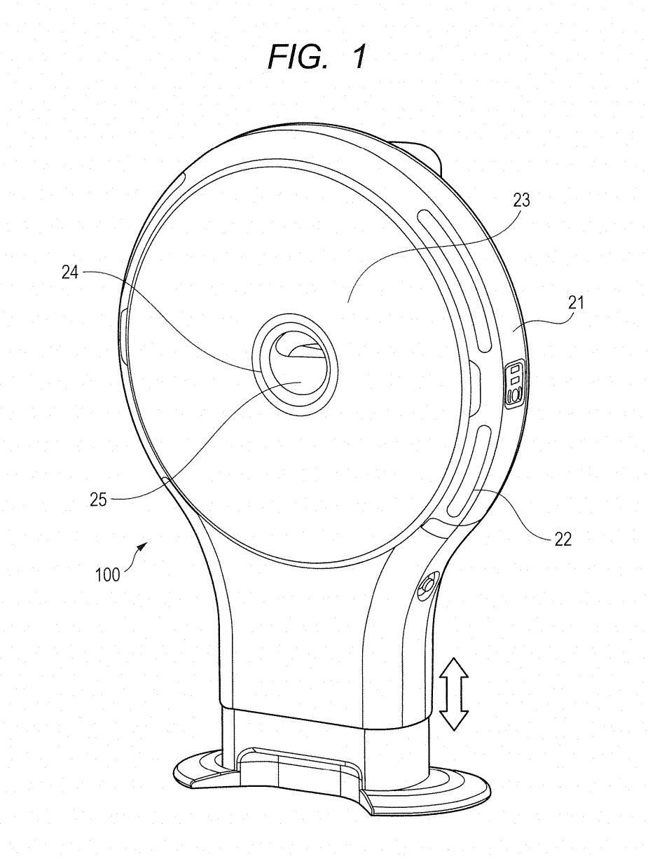

FIG. 1 is a view illustrating a medical image imaging system 100 including a radiation imaging apparatus (medical imaging apparatus) according to the present invention, as viewed from a surface on which CBCT imaging is performed. The radiation imaging system 100 is a medical image imaging system capable of performing CBCT imaging and mammogram imaging of a breast. Specifically, the radiation imaging system 100 images a medical image of a breast. An insertion hole (insertion portion) 24 from which a breast is inserted is provided at the center of a plate portion 23 which is attached to a gantry 21. A tray 25 for holding the breast is provided in the insertion hole 24. The tray 25 is coupled to the plate portion 23.

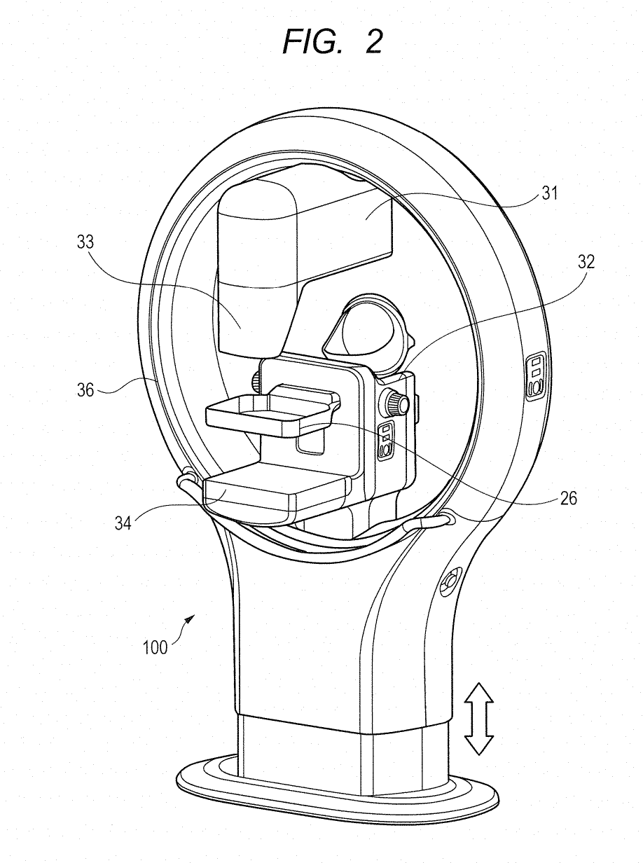

FIG. 2 is a view illustrating the radiation imaging system (medical image imaging system) 100 according to the present invention, as viewed from a surface on which mammogram imaging is performed. A CBCT X-ray tube (radiation generation unit) 31 and a mammogram X-ray tube (radiation generation unit) 33 generate radiation. A mammogram X-ray detection unit (radiation detection unit) 34 and a CBCT X-ray detection unit (radiation detection unit) 32 detect the radiation and acquire detected data of the radiation.

The CBCT X-ray tube (radiation generation unit) 31 and the CBCT X-ray detection unit (radiation detection unit) 32 are opposed to each other. The CBCT X-ray tube (radiation generation unit) 31 and the CBCT X-ray detection unit (radiation detection unit) 32 are provided on a rotatable frame (rotary portion) 36 which allows the CBCT X-ray tube 31 and the CBCT X-ray detection unit 32 to be rotated. When the rotatable frame 36 is rotated by 360 degrees, the CBCT X-ray tube 31 and the CBCT X-ray detection unit 32 are rotated by 360 degrees and CBCT imaging for breast is performed.

As illustrated in FIG. 2, the mammogram X-ray tube (radiation generation unit) 33 and the mammogram X-ray detection unit (radiation detection unit) 34 are opposed to each other. The mammogram X-ray tube (radiation generation unit) 33 and the mammogram X-ray detection unit (radiation detection unit) 34 are provided on the rotatable frame (rotary portion) 36 which allows the mammogram X-ray tube 33 and the mammogram X-ray detection unit 34 to be rotated.

Mammograph imaging is performed in a state where a breast is compressed between the mammogram X-ray detection unit 34 and a compression plate 26. The CBCT X-ray tube 31 and the mammogram X-ray tube 33 may be implemented by one X-ray tube by changing the position of the X-ray tube. The CBCT X-ray detection unit 32 and the mammogram X-ray detection unit 34 may be implemented by one X-ray detection unit by changing the position of the X-ray detection unit.

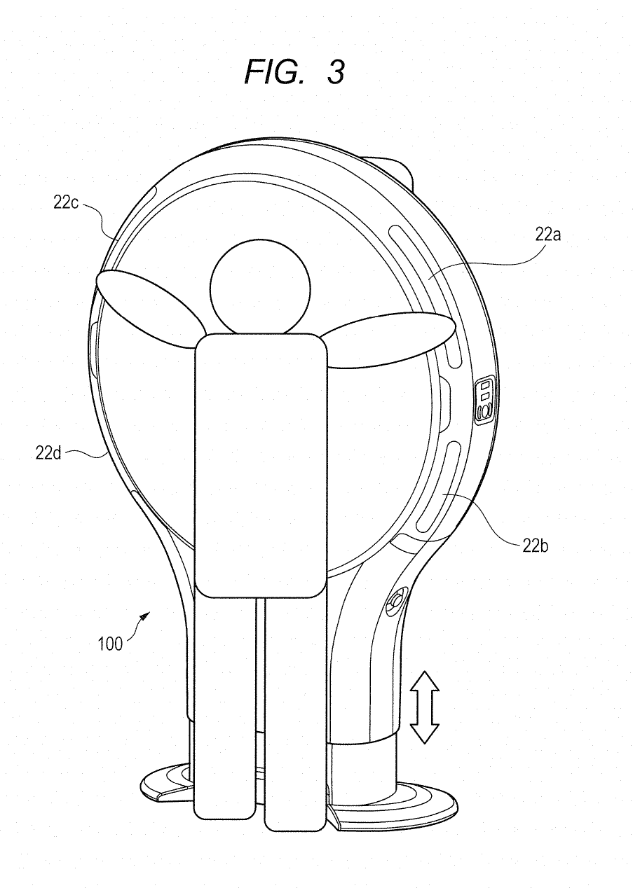

FIG. 3 is a view illustrating an imaging mode for a subject P on which CBCT imaging is performed. The subject P grips a grip portion of at least one of a plurality of grip portions 22a, 22b, 22c and 22d.

FIG. 4 is a block diagram illustrating the configuration of the radiation imaging system (medical image imaging system) 100 according to the present invention. As illustrated in FIG. 4, the radiation imaging system (medical image imaging system) 100 includes a medical image imaging apparatus that images a medical image of a breast and includes an image processing unit 102 and an imaging unit 103. The image processing unit 102 includes a determination unit 104. The imaging unit 103 includes a CBCT X-ray tube 31, a CBCT X-ray detection unit 32, a mammogram X-ray tube 33, a mammogram X-ray detection unit 34 and a measurement unit 35.

The determination unit 104 determines the insertion state of the breast based on the form of the breast inserted into the imaging area for the medical image from the insertion hole (insertion portion) 24. The form of the breast is at least one of the length, the area, the volume and the shape of the breast which is inserted into the imaging area for the medical image from the insertion hole (insertion portion) 24. Further, the determination unit 104 determines the insertion state of the breast based on the form of a non-inserted breast which is not inserted into the imaging area for the medical image from the insertion hole (insertion portion) 24. The form of the non-inserted breast is at least one of the length, the area, the volume and the shape of the non-inserted breast which is not inserted into the imaging area for the medical image from the insertion hole (insertion portion) 24.

For example, the determination unit 104 determines the insertion state of the breast based on at least one of the length, the area, the volume, the shape and the pressure of the inserted breast, which is inserted into the imaging area for the radiological image (medical image) from the insertion hole (insertion portion) 24, or the non-inserted breast which is not inserted into the imaging area. The measurement unit 35 measures at least one of the length, the area, the volume, the shape and the pressure of the breast (inserted breast or non-inserted breast). The measurement unit 35 includes a camera that images at least one of visible light, infrared light and near-infrared light.

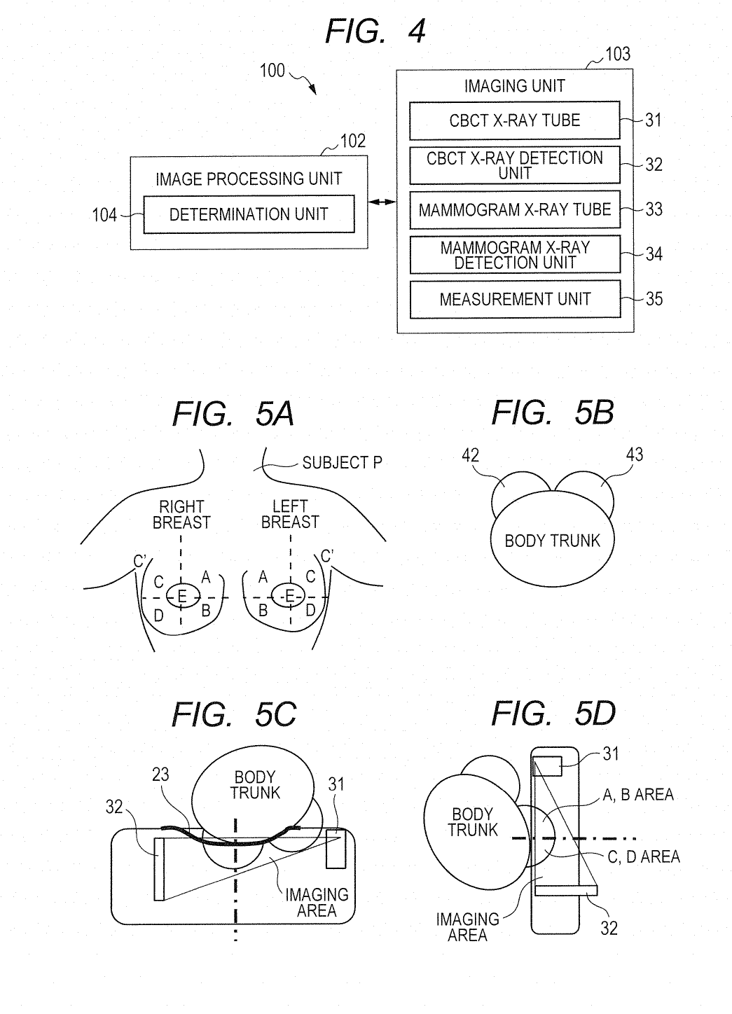

FIGS. 5A to 5D are diagrams each illustrating the insertion state of the breast during CBCT imaging. FIG. 5A is a diagram of the breast when the breast is observed from the front side of the subject P. FIG. 5A illustrates divided areas obtained by dividing the imaging area for the breast into a plurality of areas around the nipple.

The occurrence rates of breast cancer in each divided area are added up. The substantial breast cancer occurrence rates in each divided area are 15% in an A area (inside upper area), 6% in a B area (inside lower area), 50% in a C area and a C′ area (outside upper area), 11% in a D area (outside lower area), and 18% in an E area (center area) of the areola. The blind areas in the C area and the C′ area (outside upper area), which are predilection areas of breast cancer, is reduced, thereby increasing the detection accuracy of breast cancer in a breast inspection.

FIG. 5B is a diagram of the breast when the breast is observed from the leg portion side of the subject P. Anatomically, the body trunk of a human body has an elliptical section and breasts project in the radial direction from the arc-like surface of the body trunk.

FIG. 5C is a diagram illustrating a state in which the left breast of the subject P is inserted into the imaging area in the prone position type breast CBCT, as viewed from the leg portion side of the subject P. The plate portion 23 has a recess from which the breast is sufficiently inserted into the imaging range. Because the body trunk is pressed against the recess of the plate portion 23 by its own weight, the area including the thorax of the subject P can be imaged.

In other words, the prone position type breast CBCT is designed to be able to image the area including the breast bone by causing the body to be tilted by the weight of the subject so that the blind areas can be reduced. As a result, the A area, the B area, the C area, the D area and the E area can be imaged. However, tilting the body is a physical burden for the subject P, and imaging of the area including the breast bone is not preferable in terms of imaging exposure.

FIG. 5D is a diagram illustrating a state where the left breast of the subject P in the upright position is inserted into the imaging area in the upright breast CBCT, as viewed from the leg portion side of the subject P. As illustrated in FIG. 5D, when the insertion state of the breast is suitable, broad areas in the C area and D area, which are predilection areas of breast cancer, can be imaged, and thus the blind areas can be reduced.

The present invention has a feature that a suitable medical image with a reduced blind area can be acquired by determining the insertion state of a breast inserted into the imaging area for the medical image from the insertion hole (insertion portion) 24. The determination unit 104 determines the insertion state based on at least one of the length, the area, the volume, the shape and the pressure of the inserted breast in the divided areas (A area, B area, C area, C′ area, D area and E area) which are obtained by dividing the imaging area into a plurality of areas.

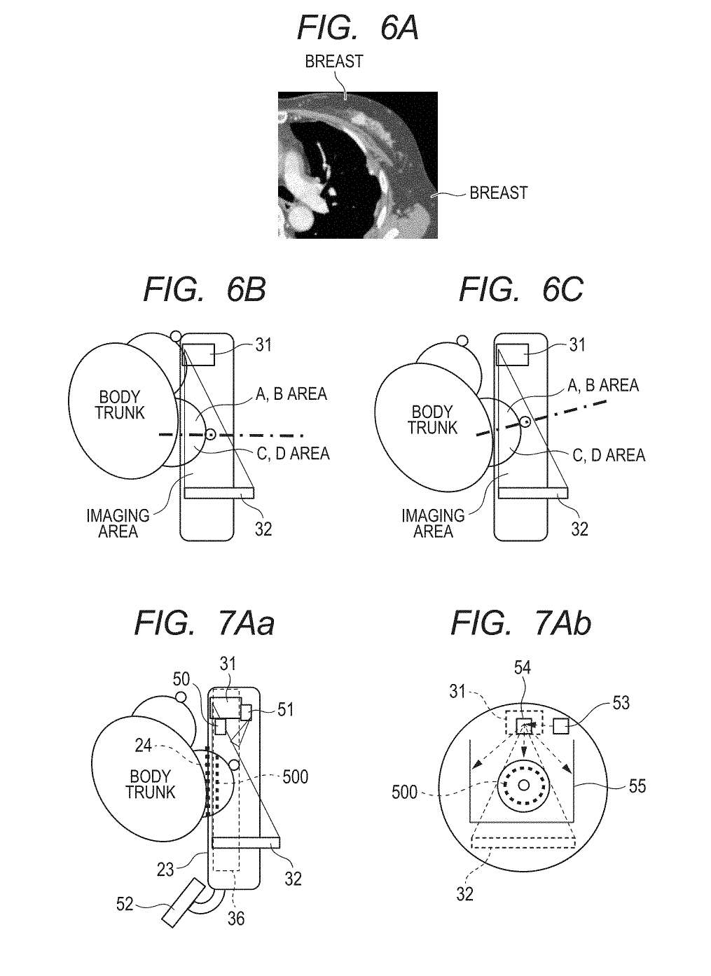

FIGS. 6A to 6C are diagrams illustrating the generation of the blind area depending on the insertion state of the breast. FIG. 6A is a radiological image of an axial section of a breast portion. As illustrated in FIG. 6A, anatomically, the section of the body trunk has an elliptical shape, and thus the breast (mammary gland) projects in the radial direction from the arc-like surface of the body trunk.

FIG. 6B and FIG. 6C are diagrams each illustrating a state where the left breast of the subject P in the upright position is inserted into the imaging area in the upright breast CBCT, as viewed from the leg portion side of the subject P. FIG. 6B illustrates a case where the left breast is inserted, while the body trunk is disposed to face the plate portion 23. Accordingly, in the case illustrated in FIG. 6B, the blind areas in the A area and B area of the left breast is small, while the blind areas in the C area and D area of the left breast is broad.

FIG. 6C illustrates a case where the left breast is inserted while the body trunk is tilted inward with respect to the plate portion 23. In the case illustrated in FIG. 6C, the blind areas in the C area and D area of the breast is small, while the blind areas in the A area and B area is broad.

In order to increase the detection accuracy of breast cancer in the breast inspection, it is efficient to reduce the blind areas in the C area and C′ area (outside upper area) which are predilection areas of breast cancer. The following embodiments are described assuming that a state where the C area and D area of the breast include small blind areas is a suitable state. That is, the insertion state illustrated in FIG. 6C is more suitable than the insertion state illustrated in FIG. 6B. In this case, however, divided areas in which the blind areas are reduced may be changed depending on the intended purpose of the breast inspection.

### First Embodiment

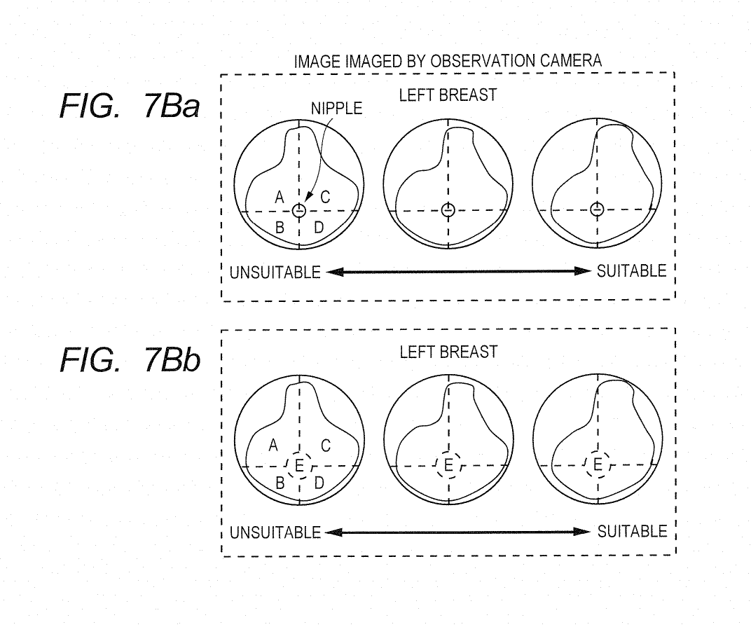

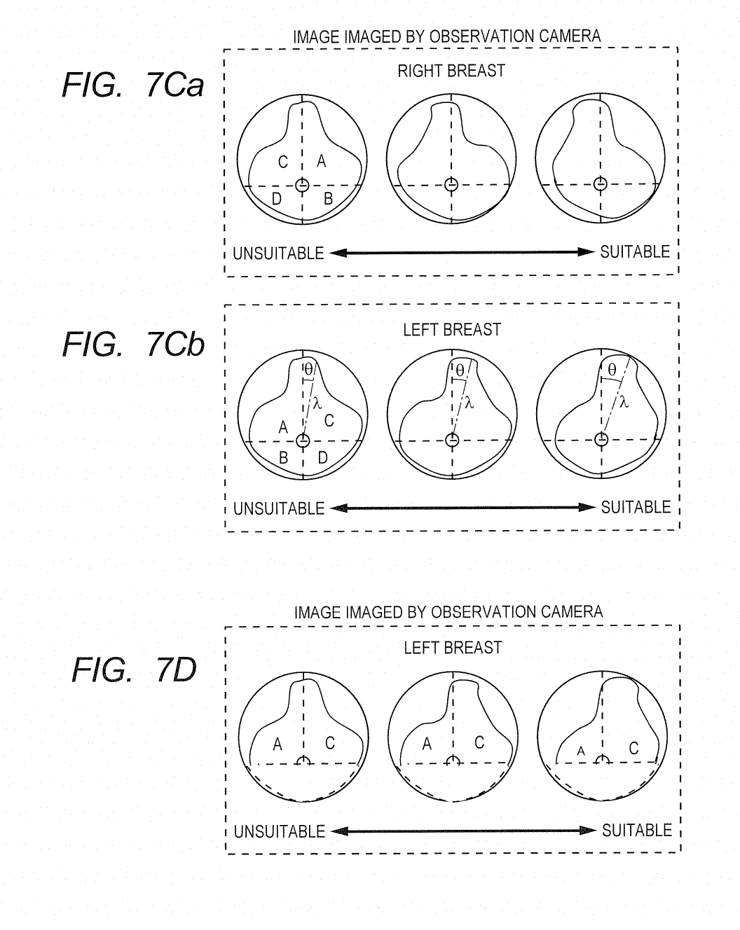

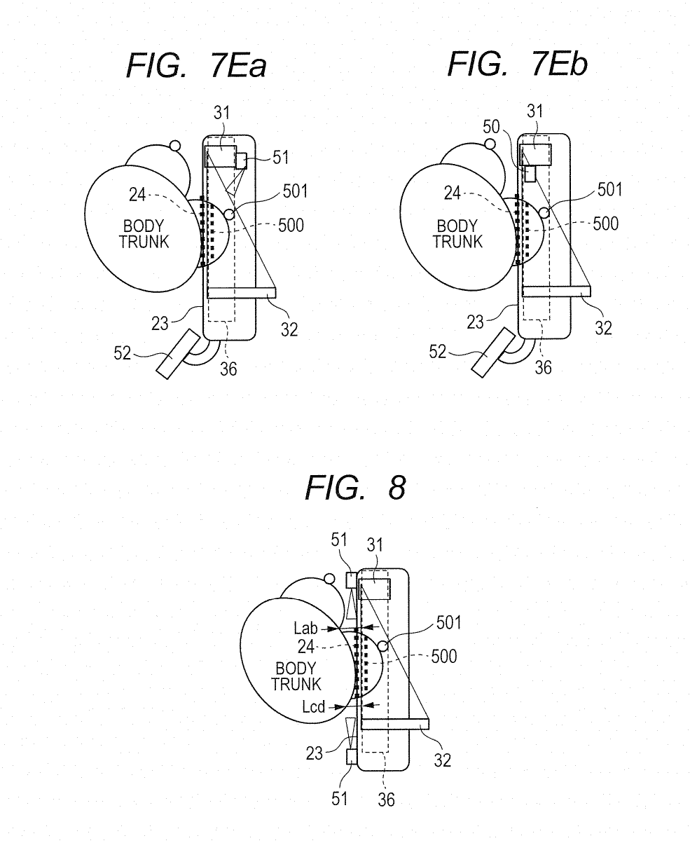

FIGS. 7Aa and 7Ab are diagrams each illustrating an example of the insertion state of the breast in the first embodiment. The radiation imaging system (medical image imaging system) 100 suitably manages blind areas in the C area and C′ area (outside upper area) which are predilection areas of breast cancer. The blind areas need to be suitably managed, except in a case where the radiological image of the whole body of the subject P is captured by a CT apparatus. In this case, to put it simply, the suitable management of blind areas indicates that the blind areas in the C area (outside upper area) and D area (outside lower area) of the breast is set to be smaller than the blind areas in the A area (inside upper area) and B area (inside lower area) of the breast.

FIG. 7Aa is a diagram illustrating a state where the left breast is inserted into the imaging area in the upright breast CBCT, as viewed from the leg portion side of the subject P. The rotatable frame 36 which is provided in the portion of the gantry 21 is provided with the CBCT X-ray tube 31 and the CBCT X-ray detection unit 32. The CBCT X-ray detection unit 32 detects the radiation emitted from the CBCT X-ray tube 31, while the rotatable frame 36 is rotated by 360 degrees, to obtain radiological image data. The radiological image data is reconfigured and calculated, to thereby generate a CT image. The plate portion 23 is coupled to the gantry 21. The insertion hole (insertion portion) 24 is provided at the center of the plate portion 23.

A projection optical system (projection portion) 50 and an observation camera 51 are installed in the vicinity of the CBCT X-ray tube 31. The projection optical system (projection portion) 50 projects the imaging area onto the inserted breast. The observation camera 51 functions as the measurement unit 35 illustrated in FIG. 4. The projection optical system 50 marks the imaging area 500 on the breast surface, and the marked imaging area are imaged by the observation camera 51 and the image processing is performed.

FIG. 7Ab is a diagram illustrating an example of a projection optical system 50 according to this embodiment. The projection optical system 50 includes a visible light (for example, red) laser 53, a Galvanometer mirror unit 54 and a reflective mirror 55.

The laser light output from the visible light laser 53 is scanned by the Galvanometer mirror unit 54, and is projected onto the breast or the reflective mirror 55. The laser light reflected on the reflective mirror 55 is projected onto the breast. The laser light allows the X-ray irradiation end (imaging area) 500 of the breast (inserted breast) inserted into the insertion hole 24 to be displayed on the breast surface. The X-ray irradiation end (imaging area) 500 is imaged by the observation camera 51. Based on the image imaged by the observation camera 51, at least one of the length, the area, the volume and the shape of the inserted breast is measured.

Note that the X-ray irradiation end (imaging area) may not be displayed on the perimeter of the breast depending on the design of the Galvanometer mirror unit 54 and the reflective mirror 55. Further, when the tray 25 is opaque with respect to visible light or the like, the X-ray irradiation end (imaging area) may not be displayed on the breast portion covered with the tray 25. The projected light from the projection optical system 50 may be at least one of visible LED light (visible light), infrared light and near-infrared light. In this case, a camera that detects visible light, infrared light and near-infrared light is selected as the observation camera 51.

FIGS. 7Ba and 7Bb are diagrams each illustrating an example in which an X-ray irradiation end (imaging area) is disposed on the perimeter of the left breast (inserted breast). Examples of insertion states from an unsuitable insertion state of the inserted breast to a suitable insertion state of the inserted breast are illustrated.

The observation camera 51 measures at least one of the length, the area, the volume and the shape of the inserted breast. The determination unit 104 determines the insertion state of the breast based on at least one of the length, the area, the volume and the shape of the inserted breast. A determination criterion can be selected from a plurality of determination criteria. The determination criterion is selected by an instruction display portion 52 illustrated in FIG. 7Aa.

The determination unit 104 determines the insertion state based on at least one of the length, the area, the volume and the shape of the inserted breast in the divided areas obtained by dividing the imaging area into a plurality of areas. The divided areas are set to at least two of a center area, an outside upper area, an inside upper area, an outside lower area and an inside lower area around the nipple.

In a first determination criterion, when the total area of the inserted breast in the C area (outside upper area) and the D area (outside lower area) is larger than the total area of the inserted breast in the A area (inside upper area) and the B area (inside lower area), the insertion state is determined as being suitable. In FIG. 7Ba, a perpendicular line and a horizontal line are indicated by dashed lines for dividing the insertion hole (insertion portion) into four divided areas. The intersection between the perpendicular line and the horizontal line represents the nipple position. The nipple position is determined by performing imaging processing (for example, color separation) on the images captured by the observation camera 51. The nipple position may be manually set.

Referring to FIG. 7Bb, the A area (inside upper area), the B area (inside lower area), the C area (outside upper area), the D area (outside lower area) and the E area (center area) are determined by the nipple position, the perpendicular line and the horizontal line. The A area (inside upper area), the B area (inside lower area), the C area (outside upper area) and the D area (outside lower area) are set in the divided areas, and areas Sa, Sb, Sc and Sd of the inserted breast in the divided areas are measured by the measurement unit 35.

(Sc+Sd)−(Sa+Sb)≧α (1)

The determination unit 104 determines whether or not Formula (1) is satisfied. When Formula (1) is satisfied, the determination unit 104 determines that the insertion state of the breast is suitable and also determines that the blind areas are suitably managed (optimized). In this case, α represents an arbitrary threshold. The threshold α for the right breast may be different from that for the left breast. Thus, the determination unit 104 determines the insertion state by comparing the predetermined threshold α with the sum and difference of at least one of the length, the area and the volume of the inserted breast in the plurality of divided areas.

The divided areas, each area, determination results, and the like are displayed on the instruction display portion (notification portion) 52. When the determination unit 104 determines that the insertion state of the breast is unsuitable, the instruction display portion (notification portion) 52 sends a notification about at least one of a warning, the insertion state, and a method for correcting the insertion state. The notification is performed by visual display or audio output.

For example, the instruction display portion (notification portion) 52 sends a notification about the direction in which the position of the subject P is changed so as to satisfy Formula (1), as the method for correcting the insertion state. When the inserted breast is the left breast, an instruction to rotate the position of the subject rightward (to tilt the body trunk inward) is sent so that the area (Sc+Sd) is larger than the area (Sa+Sb+α). When the inserted breast is the right breast, an instruction to rotate the position of the subject leftward (to tilt the body trunk inward) is notified so that the area (Sc+Sd) is larger than the area (Sa+Sb+α).

Further, the radiation imaging system (medical image imaging system) 100 may be configured to be able to start imaging on condition that the insertion state is suitable.

In a second determination criterion, when the total area of the inserted breast in the C area (outside upper area) and the D area (outside lower area) is larger than the total area of the breast in the A area (inside upper area) and the B area (inside lower area), the insertion state is determined as being suitable based on the sum and ratio of the areas of the inserted breast in the divided areas. A sum ΣS of the areas of the inserted breast in the divided areas is represented by Sa+Sb+Sc+Sd.

{(Sc+Sd)−(Sa+Sb)}/ΣS≧β (2)

The determination unit 104 determines whether or not Formula (2) is satisfied. When Formula (2) is satisfied, the determination unit 104 determines that the insertion state of the breast is suitable and also determines that the blind areas are suitably managed (optimized). In this case, β represents an arbitrary threshold. The threshold β for the right breast may be different from that for the left breast. Thus, the determination unit 104 determines the insertion state by comparing the predetermined threshold β with the sum, difference and ratio of at least one of the length, the area and volume of the inserted breast in the plurality of divided areas.

The areas Sa, Sb, Sc and Sd vary depending on the individual difference in the size of breasts. Accordingly, the individual difference in the size of breasts can be reduced based on the ratio of the sum ΣS of the areas of the inserted breast in the divided areas.

The sum (ΣS=Sa+Sb+Sc+Sd) of the areas of the inserted breast in the divided areas may be defined by a threshold. For example, when the sum ΣS of the areas of the inserted breast in the divided areas is equal to or greater than a predetermined threshold, the determination unit 104 may determine whether or not Formula (2) is satisfied. In this case, the threshold of the sum ΣS may be adjusted depending on the size of the breast. Thus, the determination unit 104 may determine the insertion state by comparing the predetermined threshold with the sum of at least one of the length, the area and the volume of the inserted breast in the plurality of divided areas.

The occurrence rate of breast cancer in each divided area may be reflected in the determination of the insertion state. For example, when the breast cancer occurrence rates in the divided areas are 15% in the A area, 6% in the B area, 50% in the C area and the C′ area, 11% in the D area and 18% in the E area, respectively, as shown in Formula (3), the insertion state may be determined using the breast cancer occurrence rate as a weighting factor.

(15*Sa+6*Sb+50*Sc+11*Sd+18*Se)/ΣS≧γ (3)

In this case, Se represents the area of the E area and γ represents an arbitrary threshold. The threshold γ for the right breast may be different from that for the left breast. The sum ΣS of the areas of the inserted breast in the divided areas is represented by Sa+Sb+Sc+Sd+Se. Thus, the determination unit 104 may determine the insertion state based on a weighting factor for a lesion occurrence rate in the divided areas. The breast cancer occurrence rate in each divided area is set by the instruction display portion (notification portion) 52 or an input unit (not illustrated).

In the same manner as described above, the divided areas, each area, determination results, the weighting factor, and the like are displayed on the instruction display portion (notification portion) 52. Further, when the determination unit 104 determines that the insertion state of the breast is unsuitable, the instruction display portion (notification portion) 52 sends a notification about at least one of a warning, the insertion state and a method for correcting the insertion state. The notification is performed by visual display or audio output.

FIGS. 7Ca and 7Cb are diagrams illustrating a case where the inserted breasts are a right breast and a left breast. When the inserted breast is switched from the right breast illustrated in FIG. 7Ca to the left breast illustrated in FIG. 7Cb, the divided areas are mirror-reversed. As a result, the inside area (area A and area B) is switched from the outside area (area C and area D). Setting of the right breast or the left breast as the inserted breast is performed by the instruction display portion (notification portion) 52 before imaging of a radiological image.

The insertion state may be determined as being suitable when the determination criterion described above is satisfied, as well as when the sum of the C area (outside upper area) and the D area (outside lower area) of the inserted breast is equal to or greater than a predetermined threshold. Also, the insertion state may be determined as being suitable when the difference between the C area (outside upper area) and the A area (inside upper area) of the inserted breast is greater than the predetermined threshold. Further, when the ratio between the C area (outside upper area) and the A area (inside upper area) of the inserted breast is larger than the predetermined threshold, the insertion state may be determined as being suitable.

The insertion state of the breast may be determined based on the sectional area or the surface area of the inserted breast in the imaging area or the divided areas, as well as on the volume of the inserted breast in the imaging area or the divided areas. Further, the insertion state of the breast may be determined based on the peripheral length or the radial length of the inserted breast in the imaging area or the divided areas.

The determination unit 104 may determine the insertion state based on the length or angle of the line from the nipple to the outer edge of the inserted breast in the divided areas. The shape of the inserted breast in the imaging area or the divided areas is approximated by a circle, an ellipse, a polygon, a sphere, an elliptical sphere, a polyhedron, or the like. Based on this shape, the insertion state of the breast may be determined.

As illustrated in FIG. 6A, the thorax of a human body has a long cylindrical shape, and the flakiness of the ellipse increases in a direction approaching the axilla. In general, in order to reduce the blind area by increasing the outside upper area (C area and C′ area), it is desirable to insert the breast into the insertion hole until the outside upper area (C area and C′ area) is brought into contact with the insertion hole (insertion portion). In this case, the length of a line from the nipple to the outer edge of the inserted breast in the outside upper area (C area and C′ area) is equal to or greater than the predetermined threshold, and the angle of the longest line from the nipple to the outer edge of the inserted breast in the outside upper area (C area and C′ area) is equal to or more than the predetermined threshold, or equal to or less than the predetermined threshold.

The alternate long and short dash line in FIG. 7Cb represents a longest inner diameter line from the nipple to the outer edge of the inserted breast in the outside upper area (C area and C′ area). Assuming that the length of an inner diameter line from the intersection between the perpendicular line and the horizontal line is represented by λ, the determination unit 104 determines whether or not Formula (4) is satisfied. Assuming that the angle from the perpendicular line to the inner diameter line is represented by an angle Θ, the determination unit 104 determines whether or not Formula (5) is satisfied.

λ≧L (4)

Θ≧Φ (5)

When Formula (4) is satisfied, the determination unit 104 determines that the insertion state of the breast is suitable and also determined that the blind areas are suitably managed (optimized). In this case, thresholds L and Φ are arbitrary thresholds. The thresholds L and Φ for the right breast may be different from those for the left breast. The determination unit 104 determines the insertion state by comparing the predetermined threshold L or the threshold Φ with the length λ or the angle Θ of a line from the nipple to the outer edge of each divided area.

In the same manner as described above, the divided areas, each area, determination results, the length of the inner diameter line, the angle of the inner diameter line, and the like are displayed on the instruction display portion (notification portion) 52. When the determination unit 104 determines that the insertion state of the breast is unsuitable, the instruction display portion (notification portion) 52 sends a notification about at least one of a warning, the insertion state, and a method for correcting the insertion state. The notification is performed by visual display or audio output.

Formula (4) or Formula (5) may be used together with any one of Formula (1) to Formula (3) (using AND condition) to determine the insertion state.

For example, when the insertion state is determined using both Formula (3) and Formula (5), the instruction display portion (notification portion) 52 sends a notification about the direction in which the position of the subject P is changed so as to satisfy Formula (5), as the method for correcting the insertion state. When the inserted breast is the left breast, an instruction to rotate the position of the subject rightward (to tilt the body trunk inward) is sent to set the angle Θ of the inner diameter line to be larger than the threshold Φ. When the inserted breast is the right breast, an instruction to rotate the position of the subject leftward (to tilt the body trunk inward) is sent to set the angle Θ of the inner diameter line to be larger than the threshold Φ.

According to this embodiment, the insertion state of the breast can be determined and the blind area can be suitably managed.

Note that the X-ray irradiation end (imaging area) is not displayed on the perimeter of the breast depending on the design of the Galvanometer mirror portion 54 and the reflective mirror 55. As illustrated in FIG. 7D, when the tray 25 is opaque with respect to visible light or the like, the X-ray irradiation end (imaging area) is not displayed in the lower area of the inserted breast covered with the tray 25.

In these cases, the determination unit 104 may determine the A area (inside upper area), the B area (inside lower area), the C area (outside upper area), the D area (outside lower area) and E area (center area) by estimating the outer edge of a missing divided area. The determination unit 104 estimates the X-ray irradiation end (imaging area) by using an arc, an elliptic arc, or the inside shape of the tray 25 so that the x-ray irradiation ends displayed by laser light are connected around the nipple.

As illustrated in FIG. 7Ea, the observation camera 51 may be installed in the vicinity of the CBCT X-ray tube 31, and the projection optical system (projection portion) 50 may be omitted. The blind areas are suitably managed by processing images captured by the observation camera 51. The observation camera 51 is a visible camera, and detects an X-ray irradiation end (imaging area) 500 and a nipple 501 by image processing. The positions of the detected X-ray irradiation end 500 and the nipple 501 are displayed on the instruction display portion 52.

The observation camera 51 illustrated in FIG. 7Ea detects the X-ray irradiation end 500 based not on marking by laser (such as visible light), but on shading of illumination light. The divided areas are determined based on the positions of the X-ray irradiation end 500 and the nipple 501, and the insertion state of the breast is determined. The detection of the X-ray irradiation end 500 based on shading of illumination light eliminates the need for the projection optical system (projection portion) 50, and thus the blind area can be suitably managed by a simple apparatus.

Also in this case, the determination unit 104 may determine the A area (inside upper area), the B area (inside lower area), the C area (outside upper area), the D area (outside lower area) and the E area (center area) by estimating the outer edge of a missing divided area.

As illustrated in FIG. 7Eb, the projection optical system (projection portion) 50 may be installed in the vicinity of the CBCT X-ray tube 31, and the observation camera 51 may be omitted. The projection optical system (projection portion) 50 projects laser (such as visible light) onto the inserted breast, and marks the imaging area 500 on the breast surface. The imaging area (or blind area) is managed by an engineer based on marking by laser (such as visible light). The X-ray irradiation end (imaging area) 500 is made clear by the projection optical system (projection portion) 50, so that the suitable management of the blind areas can be achieved by a simple apparatus.

### Second Embodiment

In the radiation imaging system (medical image imaging system) 100 of the first embodiment, the determination unit 104 determines the insertion state of the breast based on at least one of the length, the area, the volume, the shape and the pressure of the inserted breast inserted into the imaging area for the medical image from the insertion portion. On the other hand, in the radiation imaging system (medical image imaging system) 100 of a second embodiment, the determination unit 104 determines the insertion state of the breast based on at least one of the length, the area, the volume, the shape and the pressure of a non-inserted breast which is not inserted into the imaging area. Note that the descriptions of the components, functions, and operations of the second embodiment that are similar to those of the above embodiment are omitted, and differences between the second embodiment and the above embodiment are mainly described below.

FIG. 8 is a diagram illustrating an example of the radiation imaging system according to the second embodiment. As illustrated in FIG. 8, the plate portion 23 is provided with two observation cameras 51. Each observation camera 51 functions as the measurement unit 35 illustrated in FIG. 4. Blind areas are suitably managed by processing images captured by the observation cameras 51 and comparing the images. In this case, to put it simply, the suitable management of blind areas indicates that the blind areas of the breast in the C area (outside upper area) and D area (outside lower area) are set to be smaller than the blind areas of the breast in the A area (inside upper area) and B area (inside lower area).

Referring to FIG. 8, the observation cameras 51 are provided on the inside and outside of the breast. The determination accuracy of the insertion state is improved by providing the plurality of observation cameras 51 that performs imaging in various directions. Each observation camera 51 is a visible camera and measures the distance from the chest wall (chest) of the subject P to the insertion hole (insertion portion) 24. The determination unit 104 compares a distance Lab from the chest wall (chest) of the inside area (A area and B area) of the non-inserted breast to the insertion hole (insertion portion) 24 with a distance Lcd from the chest wall (chest) of the outside area (C area and D area) of the non-inserted breast to the insertion hole (insertion portion) 24.

Lab−Lcd≧δ (6)

The determination unit 104 determines whether or not Formula (6) is satisfied. When Formula (6) is satisfied, the determination unit 104 determines that the insertion state of the breast is suitable, and determines that the blind areas are suitably managed (optimized). In this case, δ represents an arbitrary threshold. The threshold δ for the right breast may be different from that for the left breast. Thus, the determination unit 104 determines the insertion state of the breast by using the distance from the chest wall (chest) to the insertion hole (insertion portion) as the length of the non-inserted breast.

The inside and outside distances Lab and Lcd, determination results, and the like are displayed on the instruction display portion (notification portion) 52. When the determination unit 104 determines that the insertion state of the breast is unsuitable, the instruction display portion (notification portion) 52 sends a notification about at least one of a warning, the insertion state, and a method for correcting the insertion state. The notification is performed by visual display or audio output.

Further, the radiation imaging system (medical image imaging system) 100 may be configured to be able to start imaging on condition that the insertion state is determined to be suitable.

According to this embodiment, the insertion state of the breast can be determined from the non-inserted breast, and the blind areas can be suitably managed.

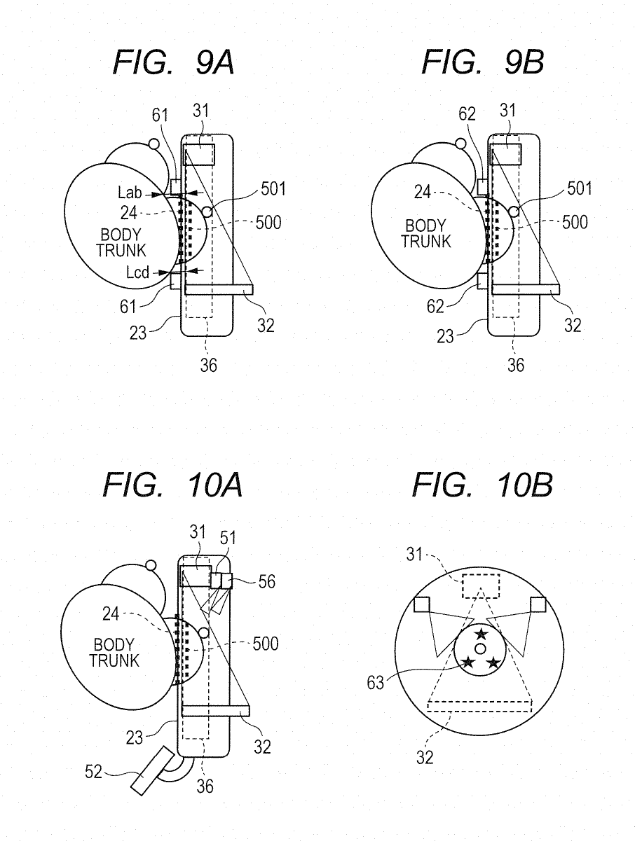

Note that a proximity sensor may be used instead of the observation camera 51. The proximity sensor functions as the measurement unit 35 illustrated in FIG. 4. As illustrated in FIG. 9A, the plate portion 23 is provided with two proximity sensors 61. The proximity sensors 61 which have strong directivity are used.

Referring to FIG. 9A, the proximity sensors 61 are provided on the inside and the outside of the breast. The determination accuracy of the insertion state is improved by providing the plurality of proximity sensors 61 that measure distances from various directions. The proximity sensor 61 measures the distance from the chest wall (chest) of the subject P to the insertion hole (insertion portion) 24. The determination unit 104 compares the distance Lab from the chest wall of the inside area (A area and B area) of the non-inserted breast to the insertion hole (insertion portion) 24 to the distance Lcd from the chest wall (chest) of the outside area (C area and D area) of the non-inserted breast to the insertion hole (insertion portion) 24.

The determination unit 104 determines whether or not Formula (6) is satisfied. When Formula (6) is satisfied, the determination unit 104 determines that the insertion state of the breast is suitable, and also determines that the blind areas are suitably managed (optimized). In this manner, the determination unit 104 determines the insertion state of the breast by using the distance from the chest wall (chest) to the insertion hole (insertion portion) as the length of the non-inserted breast.

Contact sensors (pressure sensors) may be used instead of the observation cameras 51. Each contact sensor functions as the measurement unit 35 illustrated in FIG. 4. As illustrated in FIG. 9B, the plate portion 23 is provided with two contact sensors 62.

Referring to FIG. 9B, the contact sensors 62 are respectively provided on the inside and outside of the breast. The determination accuracy of the insertion state is improved by providing the plurality of contact sensors 62 that measure pressures from various directions. Each contact sensor 62 measures the pressure applied to the plate portion 23 from the chest wall (chest) or the breast of the subject P. The determination unit 104 compares a pressure Pab applied to the plate portion 23 from the chest wall (chest) of the inside area (A area and B area) of the non-inserted breast with a pressure Pcd applied to the plate portion 23 of the chest wall (chest) of the outside area (C area and D area) of the non-inserted breast.

Pcd−Pab≧ε (7)

The determination unit 104 determines whether or not Formula (7) is satisfied. When Formula (7) is satisfied, the determination unit 104 determines that the insertion state of the breast is suitable, and determines that the blind areas are suitably managed (optimized). In this case, ε represents an arbitrary threshold. The threshold ε for the right breast may be different from that for the left breast. Thus, the determination unit 104 determines the insertion state of the breast by using the pressure applied to the plate portion 23 from the chest wall (chest) of the inside and outside areas of the non-inserted breast as the pressure of the non-inserted breast.

The contact sensor (pressure sensor) 62 may detect the pressure applied from the breast inserted into the imaging area for the medical image from the insertion hole (insertion portion) 24. In this case, the determination unit 104 determines the insertion state of the breast based on the pressure detected by the contact sensor (pressure sensor) 62.

The inside and outside pressures Pab and Pcd, determination results, and the like are displayed on the instruction display portion (notification portion) 52. When the determination unit 104 determines that the insertion state of the breast is unsuitable, the instruction display portion (notification portion) 52 sends a notification about at least one of a warning, the insertion state, and a method for correcting the insertion state. The notification is performed by visual display or audio output.

In the embodiment described above, an X-ray CT apparatus for breast imaging has been described as the radiation imaging apparatus. However, the present invention can also carry out any other medical image imaging apparatuses (breast imaging apparatuses). The present invention can be implemented as, for example, an optical-ultrasonic imaging apparatus exclusively for breast imaging, an ultrasonic imaging apparatus exclusively for breast imaging, and a PET apparatus exclusively for breast imaging. These medical image imaging apparatuses have a common feature that when a portion (for example, a breast) projecting from the body trunk is imaged, the body trunk interferes with the exterior of the imaging unit. Under the interfering state, the insertion state of the breast that is displayed as an image can be optimized.

As illustrated in FIGS. 10A and 10B, when the insertion state of the breast is determined based on the volume of the inserted breast or the non-inserted breast, a plurality of observation cameras 51 and 56 is used. The observation cameras 51 and 56 function as the measurement unit 35 illustrated in FIG. 4. The volume of the inserted breast of the non-inserted breast is calculated by generating a distance image from the plurality of observation cameras 51 and 56. When the distance image is generated, corresponding points on the image are required. Since the nipple is easily recognized on the image, the nipple is used as the corresponding point. As other corresponding points, color stickers (markers) 63 which are attached to the breast can be used.

The color stickers 63 of different colors are attached onto the breast at the positions that can be imaged by the observation cameras 51 and 56. The angle of each of the observation cameras 51 and 56 is known based on the plate portion 23, and thus the solid shape of the breast can be calculated by determining the corresponding points such as the nipple and the color stickers 63. When the number of corresponding points is small, the corresponding points may be interpolated with forecast information, or may be interpolated with a spline as a simple method. Since the nipple is also included in the corresponding points, the volumes of the A area, the B area, the C area and the D area can be calculated.

Based on the nipple position, the perpendicular line and the horizontal line, the A area (inside upper area), the B area (inside lower area), the C area (outside upper area), the D area (outside lower area) and the E area (center area) are determined. Divided areas are set in the A area (inside upper area), the B area (inside lower area), the C area (outside upper area) and the D area (outside lower area), respectively, and the volumes Va, Vb, Vc and Vd of the inserted breast in the respective divided areas are measured by the measurement unit 35.

(Vc+Vd)−(Va+Vb)≧α (8)

The determination unit 104 determines whether or not Formula (8) is satisfied. When Formula (8) is satisfied, the determination unit 104 determines that the insertion state of the breast is suitable, and determines that the blind areas are suitably managed (optimized). In this case, α represents an arbitrary threshold. The threshold α for the right breast may be different from that for the left breast. Thus, the determination unit 104 determines the insertion state by comparing the predetermined threshold α with the sum and the difference of at least one of the length, the area and the volume of the inserted breast in the plurality of divided areas.

The radiation imaging apparatus according to the embodiment of the present invention described above can achieve the suitable management of the blind areas by determining the insertion state of a breast.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-052833, filed Mar. 16, 2016, which is hereby incorporated by reference herein in its entirety.

What is claimed is:

1. A medical image imaging apparatus that images a medical image of a breast, comprising:
a determination unit configured to determine an insertion state of the breast based on a form of the breast inserted into an imaging area for the medical image from an insertion portion.

2. The medical image imaging apparatus according to claim 1, wherein
the form of the breast is at least one of a length, an area, a volume and a shape of the breast inserted into the imaging area for the medical image from the insertion portion.

3. A medical image imaging apparatus that images a medical image of a breast, comprising:
a determination unit configured to determine an insertion state of the breast based on a form of a non-inserted breast which is not inserted into an imaging area for the medical image from an insertion portion.

4. The medical image imaging apparatus according to claim 3, wherein
the form of the non-inserted breast is at least one of a length, an area, a volume and a shape of the non-inserted breast which is not inserted into the imaging area for the medical image from the insertion portion.

5. The medical image imaging apparatus according to claim 1, wherein
the determination unit determines the insertion state based on at least one of a length, an area, a volume and a shape of the breast in divided areas obtained by dividing the imaging area into a plurality of areas.

6. The medical image imaging apparatus according to claim 5, wherein
the divided areas are set to at least two of a center area, an outside upper area, an inside upper area, an outside lower area and an inside lower area around a nipple.

7. The medical image imaging apparatus according to claim 5, wherein
the determination unit determines the insertion state by comparing a predetermined threshold with at least one of a sum, a difference and a ratio of at least one of the length, the area and the volume of the breast in the plurality of divided areas.

8. The medical image imaging apparatus according to claim 7, wherein
the threshold for a right breast is different from that for a left breast.

9. The medical image imaging apparatus according to claim 5, wherein
the determination unit determines the insertion state based on a weighting factor for a lesion occurrence rate in the divided areas.

10. The medical image imaging apparatus according to claim 5, wherein
the determination unit determines the insertion state based on a length or an angle of a line from a nipple to an outer edge of the breast in the divided areas.

11. The medical image imaging apparatus according to claim 4, wherein
the determination unit determines the insertion state of the breast by setting a distance from a chest to the insertion portion as the length of the non-inserted breast.

12. The medical image imaging apparatus according to claim 1, further comprising:
a notification unit configured to send a notification about at least one of a warning, the insertion state, and a method for correcting the insertion state when the determination unit determines that the insertion state of the breast is unsuitable.

13. The medical image imaging apparatus according to claim 2, further comprising:
a measurement unit arranged to measure at least one of a length, an area, a volume, a shape and a pressure of the breast.

14. The medical image imaging apparatus according to claim 13, wherein
the measurement unit includes a camera arranged to image at least one of visible light, infrared light and near-infrared light.

15. A medical image imaging apparatus that images a medical image of a breast, comprising:
a pressure sensor arranged to detect a pressure applied from a breast inserted into an imaging area for the medical image from an insertion portion; and
a determination unit configured to determine the insertion state of the breast based on the pressure detected by the pressure sensor.

16. The medical image imaging apparatus according to claim 1, further comprising:
a projection unit arranged to project the imaging area onto the inserted breast.

17. An insertion state determination method, comprising:
detecting a form of a breast inserted into an imaging area for a medical image of a breast from an insertion portion of a medical image imaging apparatus configured to image the medical image of the breast; and
determining an insertion state of the breast based on the form of the breast.

18. An insertion state determination method comprising:
detecting a form of a non-inserted breast which is not inserted into an imaging area for a medical image of a breast from an insertion portion of a biometrical imaging apparatus configured to image the medical image of the breast; and
determining an insertion state of the breast based on the form of the non-inserted breast.

19. An insertion state determination method, comprising:
detecting a pressure applied from a breast inserted into an imaging area for a medical image of the breast from an insertion portion of a medical image imaging apparatus configured to image the medical image of the breast; and
determining an insertion state of the breast based on the detected pressure.
